SEQUENCE LISTING (2 sequences)

SEQ ID NO: 1 - DNA, 390 bp, Bacillus subtilis (strain HE)
  misc_feature (47)..(47): n is a, c, g, or t
cggatggttgtttgaaccgcatggttcaaacataaaaggtggtttcngctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggnaatacgtaggtggcaagcgttgtc

SEQ ID NO: 2 - DNA, 450 bp, Bacillus licheniformis (strain PA)
  misc_feature (369)..(369): n is a, c, g, or t
caccgcggcatgctgatccgcgattactagcgattccagcttcacgcagtcgagttgcagactgcgatccgaactgagaacagatttgtgggattggcttagcctcgcggcttcgctgccctttgttctgcccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggaagccctatctctagggttgncagaggatgtcaagacctggtaaggttcttcgcgttgcttcgaattaaaccacatgctccaccgcttgtgcgggcccccgt

(19) United States
(12) Patent Application Publication
(10) Pub. No.: US 2005/0271643 A1
(43) Pub. Date: Dec. 8, 2005

(54) Bacterial strains, compositions including same and probiotic use thereof

(76) Inventors: Iryna Sorokulova, Auburn, AL (US); Irina Ossipova, Moscow (RU)

Correspondence Address: Martin MOYNIHAN;c/o ANTHONY CASTORINA, SUITE 207, 2001 JEFFERSON DAVIS HIGHWAY, ARLINGTON, VA, 22202, US

(21) Appl. No.: 10/916,646

(22) Filed: Aug. 12, 2004

Related U.S. Application Data
(60) Provisional application No. 60/494,786, filed on Aug. 14, 2003.

(51) Int. Cl. A61K 45/00; C12N 1/20

(52) U.S. Cl. 424/93.462; 435/252.31

(57) ABSTRACT

A bacterial co-culture is provided. The bacterial co-culture comprises a first bacterial strain having all the identifying characteristics of Bacillus licheniformis PA (ATCC Deposition No: PTA-53 11) and a second bacterial strain having all the identifying characteristics of Bacillus subtilis HE (ATCC Deposition No: PTA-5310)

This application claims the benefit of priority of U.S. provisional patent application No. 60/494,786, filed Aug. 14, 2003.

### FIELD AND BACKGROUND OF THE INVENTION

The present invention relates to novel bacterial strains, compositions including same and methods of using such strains in probiotic treatment of gastrointestinal disorders, such as diarrhea.

Probiotics are defined as living organisms, which exert a positive effect on a host gastro-intestinal (GI) system. The most commonly used probiotics are strains of the lactic acid bacteria (LAB), particularly those classified to the Lactobacillus, Lactococcus, and Enterococcus genera.

It is well known that during periods of low resistance (e.g., stress or disease, at birth or following antibiotic treatments) undesirable microorganisms are able to proliferate in the gastrointestinal tract. Thus, maintaining a normal, healthy flora of microorganism in the gastrointestinal (GI) tract is critical during stressful periods.

The goal of probiotic therapy is to increase the number and activity of health-promoting microorganisms until normal GI flora can be reestablished.

Several mechanisms responsible for the protective action of probiotics have been proposed. These include, (i) the production of inhibitory substances (e.g., antibiotics, organic acids, hydrogen peroxide and bacteriocins) which may reduce cell viability, affect bacterial metabolism and reduce toxin production; (ii) blocking of adhesion sites by competitive inhibition of bacterial adhesion sites on intestinal epithelial surfaces [Conaway (1987) J. Dairy Sci. 70:1-12; Goldin (1992) Dig. Dis. Sci. 37:121-128; Kleeman and Klaenhammer (1982) J. Dairy Sci. 1982;65:2063-2069]; (iii) competition for nutrients; (iv) degradation of toxin receptors, which is the postulated mechanism by which S. boulardii protects animals against C. difficile intestinal disease through the degradation of the toxin receptor on the intestinal mucosa [Castagliuolo (1996) Infect. Immun. 64:5225-5232; Castagliuolo Infect. Immun. (1999) 67:302-307 Pothoulakis (1993) Gastroenterology 104:1108-1115]; (v) and stimulation of non-specific immunity [Fukushima Int. J. Food Microbiol. (1998) 42:39-44; Link-Amster FEMS Immunol. Med. Microbiol. (1994) 10:55-63; Malin Ann. Nutr. Metab. (1996) 40:137-145].

As is further detailed below, numerous studies investigated the possible use of probiotics in treatment and prevention of various intestinal and extraintestinal disorders.

Acute diarrhea—The major manifestation of enteric infection is diarrhea. Although rehydration therapy is efficacious in many instances, its acceptance is low since it neither reduces stool frequency nor does it shorten the duration of diarrhea. Furthermore, it is difficult to implement in small children.

Probiotic treatment of diarrhea has been attempted with limited success. S. boulardii administration to pediatric patients effected in a double-blind placebo-controlled study caused significant reduction in stool frequency [Cetina Sauri (1994) Extrait Ann Pediatrie 41:6]. On the other hand, no therapeutic efficacy of Streptococcus faecium in acute watery diarrhea caused by Vibrio cholera and enterotoxic E. coli was observed [Mitra (1990) 99:1149-52].

Traveller's diarrhea—Traveller's diarrhea is a common syndrome affecting healthy travelers not only in developing countries but also in the western world. The incidence of Traveller's diarrhea ranges from 20 to 50% depending on the origin and destination of the traveler as well as the mode of travel. The diarrhea is self-limiting but even minor attacks can interrupt a holiday, causing inconvenience and discomfort. Various infectious agents have been described as the cause of Traveller's diarrhea. Toxin-producing Escherichia coli are the most commonly isolated organism.

Probiotics have been shown to have beneficial effect in preventing some forms of Traveller's diarrhea if live acid bacteria are administered during the risk period [Du Pont (1993) New Eng. J. Med. 328:1821-7; Van der Waij (1982) J. Antimicrob. Ther. 10:263-70; Oksanen (1990) Ann. Med. 22:53-6; Salminen (1992) 10:227-38; Black (1989) Travel Med. 8:333-5; Katelaris (1995) 41:40-7; Kollaritsch (1990) 74-82].

Antibiotic associated diarrhea—Mild or severe episodes of diarrhea are most common side effects of antibiotic therapy. It is well established that the normal microflora can be suppressed during microbial therapy and the consequent microbial deficiency may be replaced by opportunistic or pathogenic strains [Gismondo (1995) Chemotherapy 41:281-8]. Changes in microflora may also encourage the emergence of resistant strains and at least a third of antibiotic associated diarrhea is due to Clostridium difficile.

It has been suggested that probiotics can be used to restore and replace the normal intestinal flora. In particular, probiotics can be used in high risk patients such as the elderly, hospitalized or immunocompromised. Several clinical trials have used S. boulardii, Lactobacillus spp. and Bifidobacterium spp. in antibiotic associated diarrhea. Thus, for example, administration of S. boulardii to hospitalized patients reduced the incidence of antibiotic associated diarrhea by 50% [Surawicz (1989) Gastroenterology 96:981-8; McFarland (1995) Am. J. Gastroenterol 90:439-48]. Alternatively, administration of Lactobacillus GG to patients with C. difficile colitis halted diarrhea with no relapse incidents [Gorbach (1987) Lancet 2:1519-22].

HIV-associated diarrhea—Diarrhea is a very serious consequence of human immunodeficiency virus (HIV) infection. The etiology of this diarrhea is frequently unknown and there are no effective treatment modalities. However, S. boulardii was recently used to treat 33 HIV patients with chronic diarrhea (Born et al. Dtsch. Med. Wochenschr. (1993);118:765, Saint-Marc et al. (1991) Ann. Med. Intern. 142:64-65). In these double-blind studies, 56% of patients receiving S. boulardii had resolution of diarrhea compared with only 9% of patients receiving placebo.

Sucrase-isomaltase deficiency—Sucrase-isomaltase deficiency is the most frequent primary disaccharidase deficiency in humans. It is an inherited condition that leads to malabsorption of sucrose. The resulting bacterial fermentation of the sucrose leads to an accumulation of hydrogen in the colon, producing diarrhea, abdominal cramps and bloating. A sucrose-free diet causes a disappearance of symptoms. However, not all patients will follow such a diet. Harms et al. [(1987) N. Engl. J. Med. 316:1306-1309] used Saccharomyces cerevisiae to treat eight children with sucrase-isomaltase deficiency. It was demonstrated that in children given sucrose followed by S. cerevisiae, there was an improvement in both their hydrogen breath test and gastrointestinal symptoms, which may be caused by enzymatic complementation with S. cerevisiae enzymes.

Rotavirus diarrhea—Rotaviruses are a significant cause of infant morbidity and mortality, particularly in developing countries [Majamaa et al. (1995) J. Pediatr. Gastroenterol. Nutr 20:333-338, Middleton et al. (1977) Am. J. Dis. Child. 131:733-737]. The principal means of treatment is oral rehydration, although an effective vaccine that should decrease dramatically the health impact of rotavirus infections has recently become available.

Lactobacillus has demonstrated some promise as a treatment for rotavirus infection [Isolauri et al. (1994) Dig. Dis. Sci.39:2595-2600, Kaila et al. (1992) Pediatr. Res. 32:141-144, Majamaa et al. (1995) Supra ). Isolauri et al. (1991) treated 74 children (ages 4-45 mo) with diarrhea with either Lactobacillus GG or placebo. Approximately 80% of the children with diarrhea were positive for rotavirus. The investigators demonstrated that the duration of diarrhea was significantly shortened (from 2.4 to 1.4 d) in patients receiving Lactobacillus GG. The effect was even more significant when only the rotavirus-positive patients were analyzed.

Inflammatory bowel disease—Two inflammatory bowel diseases including Crohn's disease and ulcerative colitis with unknown etiologies, are related to disturbances of the intestinal microbial flora [Fabia et al. (1993) Digestion 54:248-255]. Crohn's disease is an idiopathic inflammatory bowel disease that occurs from the mouth to the anus, although the terminal ileum is most common site of disease. The most common clinical manifestation of ulcerative colitis is an inflammation of the colon. No specific treatment is available for either disease. The Nissle strain of nonpathogenic E. coli (serotype O6:K5:H1) was examined for its ability to prevent relapses of ulcerative colitis [Kruis (1997) Aliment. Pharmacol. Ther. 11:853-858]. Preliminary results look promising and suggest that this may be another option for maintenance therapy of ulcerative colitis.

Constipation—Constipation is a common condition occurring with increasing frequency in advanced age. Within the UK, for example, it is estimated that three million GI consultations relate to constipation every year [Robinson, Constipation: causes and cures, Nurs Times. Jun. 24-30, 2003;99(25):26-7].

The problem of constipation is often associated with the changes in gastrointestinal microflora [Colum Dunne Inflammatory Bowel Diseases (2001) 7:136-145]. Probiotic treatment has been suggested to improve intestinal motility, reduce fecal enzyme activity and to be effective in relief from constipation [Ouwehand et al Annals of Nutrition and Metabolism (2002) 46:159-162).

Pouchitis—Pouchitis is a complication of ileal reservoir surgery occurring in 10-20% of the patients who undergo surgical treatment for chronic ulcerative colitis. Bacteria overgrow in the pouch, resulting in degradation of the mucus overlaying the epithelial cells. This results in inflammation and symptoms that include bloody diarrhea, lower abdominal pain and fever. Lactobacillus GG os proposed to be an effective therapeutic agent for pouchitis because it does not demonstrate mucus-degrading properties [Ruseler-Van Embden et al. (1995) Microecol. Ther. 23:81-88].

Carcinogenesis—Evidence is accumulating that the normal intestinal flora can influence carcinogenesis by producing enzymes that activating carcinogens. These enzymes include glycosidase, β-glucuronidase, azoreductase and nitroreductase. Apparently, selected microorganisms may protect the host from this carcinogenic activity [Orrhage et al. (1994) Mutat. Res. 311:239-248; Rowland and Grasso (1975) Appl. Microbiol. 29:7-12]. Human subjects receiving either L. acidophilus or L. casei have reduced levels of enzymes that convert precarcinogens to carcinogens in their fecal specimens [Hayatsu and Hayatsu (1993) Appl. Microbiol. 29:7-12; Lee and Salminen (1995) Trends Food Sci. Technol. 6:241-245; Lidbeck et al. (1992) Eur. J. Cancer Prev. (1992) 1:341-353].

Enteral feeding associated diarrhea—Patients receiving nasogastric tube feeding frequently develop diarrhea. The mechanism of the diarrhea is not known, but it is postulated that enteral feeding causes changes in normal flora that result in altered carbohydrate metabolism and subsequent diarrhea. Two separate studies, both placebo-controlled and double blind, demonstrated a significant reduction in diarrhea in these patients when they were given S. boulardii [Bleichner et al. (1997) Intensive Care Med. 23:517-523, Tempe et al. (1983) Sem. Hop. 59:1409-1412].

Uro-genital tract diseases—Uterine infections and infections of the cervix, vagina and vulva commonly occur in human beings and domestic animals, especially following birth. Typical infecting organisms of the endometrium (i.e., uterine mucosa) and contiguous mucosal surfaces in the lower genital tract include, for example, β-hemolytic streptococci, Candida albicans, Klebsiella pneumoniae, coliform bacteria including Escherichia coli, Corynebacterium pyogenes and C. vaginale, various Campylobacter or Trichomonas species such as T. vaginalis, and the like (see U.S. Pat. No. 5,667,817).

Other urogenital pathogens include but are not limited to Chlamydia trachomatis, Neisseria gonorrhoeae, herpes simplex virus, HIV, papillomavirus and Treponema pallidum.

Bacterial Vaginitis (BV) can lead to complications in pregnancies, causing premature rupture of the membranes, premature birth, or the death of the fetus or newborn. Premature rupture of the membranes can also be associated with BV, urinary tract infections, group B streptococcal infections, and the presence of organisms such as ureaplasma and mycoplasma in the urogenital tract

Once investigation has ruled out complicated underlying causes, the only therapeutic option is antimicrobial agents. In many cases, this is effective at clearing infection. However, recurrences, side effects, and secondary infections are frequent. Coinciding with infection is a disruption of the normal commensal microflora in the vagina, primarily a loss of lactobacilli. The use of probiotics has been shown to be beneficial in treating urogenital tract diseases as described by Reid FEMS Immunol Med Microbiol. (2001) February;30(1):49-52.

Respiratory diseases—The upper respiratory tract may harbor potential pathogenic bacteria including but not limited to, Staphylococcus aureus, Streptococcus pneumoniae, beta-hemolytic streptococci and Haemophilus influenza. A number of reports suggest that regular intake of probiotics can reduce the number of potential pathogenic bacteria in the upper respiratory tract [Roos and Kolm (2002) Curr. Infect. Dis. Rep. 4:211-216]. Guarino and co-workers [Gastroenterol Int 1998;11(suppl):91] described a significant reduction in the severity of pneumonia in children with cystic fibrosis treated with Lactobacillus GG compared with a placebo group. Ribeiro and Vanderhoof [J Pediatr Gastroenterol Nutr 1998;26:561] also showed that the introduction of probiotics to children who attended daycare centers reduced the incidence of respiratory disease. A number of mechanisms may explain the effect of probiotics on respiratory diseases. Mack et al [Am J Physiol 1999;276:G941-50] showed up-regulation of mucin genes in cell culture systems by L. plantarum. Lactobacillus GG appears to selectively stimulate the antibody reaction to both rotavirus and rotavirus vaccine, a property not shared by most other species of lactobacilli. Finally, Jung and co-workers [FASEB J 1999;13:A872] showed that Lactobacillus GG produced a better antibody response to typhoid vaccine in adults treated with Lactobacillus GG than in a placebo group [Jung et al. FASEB J 1999;13:A872].

Rheumatoid arthritis—It is appreciated that the inflammation associated with rheumatoid arthritis might be modulated by consuming probiotics [Malin (1996) Br J Rheumatol;35:689-94]. Normal processing of antigens absorbed through an inflamed and permeable gastrointestinal tract might serve as a link between inflammatory diseases of the gut and extraintestinal inflammatory disorders. Modulation of the immune system or changed gut permeability as a result of consuming probiotics might eventually become an important primary or adjunctive therapy in some of these disorders.

Allergies—Allergic diseases have increased over the past two or three decades probably due to reduced microbial stimulation associated with the western world lifestyle (i.e., improvement of hygiene and reduced family size). These changes in lifestyle may have induced alterations in the microflora composition, resulting in reduced stimulation of the immune system. Is hypothesized that intestinal infections are important in the neonatal stage in order to shift the default reactivity of cytokine Th2 towards that of cytokine Th1, thus reducing the incidence of allergy.

A major characteristic of the microflora harbored by individuals with higher prevalence of atopic disease is the decrease in Lactobacillus and Eubacterium combined with higher counts of Clostridium ssp [Biorksten et al. Clin. Exp. Allergy (1999) 29:342-346]. Thus, it has been attempted to introduce probiotics to correct the dysbalance of the microflora to thereby treat atopic diseases.

Recently the positive activities of certain probiotics for the prevention of atopic eczema have been reported [Isolauri (2001) Am J Clin Nutr 73:1142S-1146S]. In the management of atopic eczema two different bacteria, Bififobacterium lactis and Lactobacillus rhamnosus GG, have proven efficient in a controlled study [Kalliomaki (2001) Lancet 357:1076-1079].

Although the effectiveness of probiotic therapy has been demonstrated by numerous studies and thus is now accepted as suitable therapy for a number of disorders, probiotic treatment can lead to a number of side effects including systemic infections, deleterious metabolic activities, excessive immune stimulation in susceptible individuals and gene transfer [Marteau (2001) Safety aspects of probiotic products. Scand. J. Nutr., 45, 1, 22-24]. For example, two cases of L. rhamnosus were traced to possible probiotic consumption [Rautio (1999) Clin. Infect. Dis. 28:1159-60; Mackay (1999) Clin. Microbiol. Infect. 5:290-292]. Thirteen cases of Saccharomyces fungemia were caused by vascular catheter contamination [Hennequin (2000) Eur. J. Clin. Microbiol. Infect. Dis. 19:16-20] and Bacillus infections linked to probiotic consumption all in patients with underlying disease [Spinosa (2000) Microb. Ecol. Health Dis. 12:99-101; Oggioni (1998) J. Clin. Microbiol. 36:325-326]. Alternatively, Enterococcus is emerging as an important cause of nosocomial infections and isolates are increasingly vancomycin resistant.

There is, thus, a widely recognized need for and it would be highly advantageous to have, probiotic bacterial strains, which are devoid of the above limitations.

### SUMMARY OF THE INVENTION

According to one aspect of the present invention there is provided a biologically pure culture of a bacterial strain having all the identifying characteristics of the Bacillus subtilis HE strain (ATCC Deposition No: PTA-5310).

According to another aspect of the present invention there is provided a biologically pure culture of a bacterial strain having all the identifying characteristics of the Bacillus licheniformis PA strain (ATCC Deposition No: PTA-5311).

According to yet another aspect of the present invention there is provided a bacterial co-culture comprising a first bacterial strain having all the identifying characteristics of Bacillus licheniformis PA (ATCC Deposition No: PTA-5311) and a second bacterial strain having all the identifying characteristics of Bacillus subtilis HE (ATCC Deposition No: PTA-5310).

According to still another aspect of the present invention there is provided a bacterial co-culture comprising at least two bacterial strains including a Bacillus licheniformis strain and a Bacillus subtilis strain, the bacterial co-culture exhibiting a higher anti-pathogenic activity than a Biosporin culture.

According to further features in preferred embodiments of the invention described below, the Bacillus licheniformis strain is Bacillus licheniformis PA (ATCC Deposition No: PTA-5311) and the Bacillus subtilis strain is Bacillus subtilis HE (ATCC Deposition No: PTA-5310).

According to an additional aspect of the present invention there is provided a composition comprising a therapeutically effective amount of a first bacterial strain having all the identifying characteristics of Bacillus licheniformis PA (ATCC Deposition No: PTA-5311) and/or a second bacterial strain having all the identifying characteristics of Bacillus subtilis HE (ATCC Deposition No: PTA-5310) and a pharmaceutically acceptable carrier.

According to still further features in the described preferred embodiments the composition including at least 10³ viable bacteria cells per gram.

According to still further features in the described preferred embodiments the lo composition including at least 10⁶ viable bacteria cells per gram.

According to still further features in the described preferred embodiments the composition including at least 10¹⁰ viable bacteria cells per gram.

According to still further features in the described preferred embodiments the composition further comprising a probiotic microorganism selected from the group consisting of a yeast cell, a mold and a bacterial cell.

According to still further features in the described preferred embodiments the composition further comprising an antibiotic.

According to still further features in the described preferred embodiments the composition further comprising an antifungal agent.

According to an additional aspect of the present invention there is provided a food additive or supplement comprising an effective amount of a first bacterial strain having all the identifying characteristics of Bacillus licheniformis PA (ATCC Deposition No: PTA-5311) and/or a second bacterial strain having all the identifying characteristics Bacillus subtilis HE (ATCC Deposition No: PTA-5310) and a carrier suitable for human consumption.

According to still further features in the described preferred embodiments the carrier is a colonization carrier.

According to still further features in the described preferred embodiments the colonization carrier is selected from the group consisting of a saccharide, a modified saccharide and a combination thereof.

According to yet an additional aspect of the present invention there is provided a feed additive or supplement comprising an effective amount of a first bacterial strain having all the identifying characteristics of Bacillus licheniformis PA (ATCC Deposition No: PTA-5311) and/or a second bacterial strain having all the identifying characteristics of Bacillus subtilis HE (ATCC Deposition No: PTA-5310) and a carrier suitable for animal consumption.

According to still further features in the described preferred embodiments the carrier is selected from the group consisting of limestone, saccharides and wheat midds.

According to still an additional aspect of the present invention there is provided a foodstuff comprising an effective amount of a first bacterial strain having all the identifying characteristics of Bacillus licheniformis PA (ATCC Deposition No: PTA-5311) and/or a second bacterial strain having all the identifying characteristics of Bacillus subtilis HE (ATCC Deposition No: PTA-5310).

According to still further features in the described preferred embodiments the foodstuff being a fermented milk product.

According to a further aspect of the present invention there is provided a method of treating or preventing a gastrointestinal disorder, the method comprising administering to a subject in need thereof a therapeutically effective amount of a first bacterial strain having all the identifying characteristics of Bacillus licheniformis PA (ATCC Deposition No: PTA-5311) and/or a second bacterial strain having all the identifying characteristics of Bacillus subtilis HE (ATCC Deposition No: PTA-5310).

According to yet a further aspect of the present invention there is provided an article-of-manufacture comprising packaging material and a composition identified for treating or preventing a gastrointestinal disorder being contained within the packaging material, the composition including, as an active ingredient, a first bacterial strain having all the identifying characteristics of Bacillus licheniformis PA (ATCC Deposition No: PTA-5311) and/or a second bacterial strain having all the identifying characteristics of Bacillus subtilis HE (ATCC Deposition No: PTA-5310).

According to still a further aspect of the present invention there is provided a method of treating or preventing a disorder which may be treated or prevented by probiotics, the method comprising administering to a subject in need thereof a therapeutically effective amount of a first bacterial strain having all the identifying characteristics of Bacillus licheniformis PA (ATCC Deposition No: PTA-5311) and/or a second bacterial strain having all the identifying characteristics of Bacillus subtilis HE (ATCC Deposition No: PTA-5310).

According to still further features in the described preferred embodiments the first or the second bacterial strain is provided in a sporolated form.

According to still further features in the described preferred embodiments the first or the second bacterial strain is provided in a lyophilized form.

According to still further features in the described preferred embodiments the administering is effected at a concentration of the first bacterial strain and/or the second bacterial strain between 10⁸ and 10¹⁰ viable cells in one dose.

According to still further features in the described preferred embodiments the disorder is selected from the group consisting of appendicitis, autoimmune disorders, multiple sclerosis, Alzheimer's disease, rheumatoid arthritis, coeliac disease, diabetes mellitus, organ transplantation, periodontal disease, urogenital diseases, sexually transmitted disease, HIV infection, HIV replication, surgical associated trauma, surgical-induced metastatic disease, sepsis, weight loss, anorexia, fever control, cachexia, wound healing, ulcers, gut barrier function, allergy, asthma, respiratory disorders, rhinovirus-associated diseases, circulatory disorders, coronary heart disease, anaemia, disorders of the blood coagulation system, renal disease, disorders of the central nervous system, hepatic diseases, constipation, ischaemia, nutritional disorders, osteoporosis, endocrine disorders, epidermal disorders, psoriasis, anthrax and acne vulgaris.

The present invention successfully addresses the shortcomings of the presently known configurations by providing novel bacterial strains, compositions including same and probiotic use thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are photomicrographs illustrating the morphology of Bacillus licheniformis PA (FIG. 1a) and Bacillus subtilis HE (FIG. 1b), 18 hours following cultivation on nutrient agar at 37° C.×1000 magnifications are shown.

### DESCRIPTION OF THE PREFERRED EMBODIMENTS

The present invention is of bacterial strains and compositions including same, which can be used for probiotic treatment of gastrointestinal disorders, such as diarrhea.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The gastrointestinal microflora is important for maintaining gastrointestinal tract function and overall physiological health of human beings and animals. However, during periods of low resistance, undesirable microorganisms (i.e., pathogens) are able to proliferate in the gastrointestinal tract, thereby replacing the normal protective intestinal flora leading to the emergence of severe gastrointestinal symptoms.

Attempts to modify the structure and metabolic activities of indigenous bacteria have been effected primarily with probiotics, which are live microorganisms, which exert a positive effect on a host gastro-intestinal (GI) system. To date, the best-known probiotics are the lactic acid-producing bacteria (i.e., Lactobacilli and Bifidobacteria), which are widely utilized in yogurts and other dairy products. These probiotic organisms are non-pathogenic and non-toxigenic, retain viability during storage, and survive passage through the stomach and small intestine.

The probiotic Biosporin is a culture of aerobic sporulating bacteria of the Bacillus genus including Bacillus subtilis 3 and Bacillus licheniformis 31. Biosporin is characterized by antagonistic activity against a wide range of pathogenic and conditionally pathogenic microorganisms including antibiotic resistant microorganisms [e.g., Salmonella spp., Shigella spp, enteropathogenic E. coli, Proteus spp., Klebsiella spp., S. aureus, Campylobacter spp., Helicobacter spp., Yersinia spp., Candida spp.]. Importantly, Biosporin exhibits higher therapeutic efficacy as compared to other probiotic preparations, such as lactobacilli, and only minimal cytotoxicity as high dosage administration does not result in any negative effects on the host, such as systemic infections and deleterious metabolic activities [Sorokulova (1997) Mikrobiol Z. 59(6):43-9; Smirnov et al. (1994) Likarska sprava, 5-6, 133-138; Gracheva et al 1996) Zh. Microbiol. (Moscow) 1, 75-77; Osipova et al. (1998) Zh Mikrobiol Epidemiol Immunobiol., 6, 68-70]. Furthermore, Biosporin is the only probiotic culture known to date, which is effective against Campylobacter pathogens [Sorokulova et al. (1997) J. Travel. Med. 4:167-170].

While reducing the present invention to practice and while searching for bacterial strains with improved probiotic activity, the present inventor(s) uncovered novel bacterial strains, which exhibit superior probiotic functions as compared to a Biosporin culture.

As is illustrated in the Examples section, which follows, the bacterial strains of the present invention were uncovered through selection of a Biosporin culture for improved casein decomposition and lysozyme production activities.

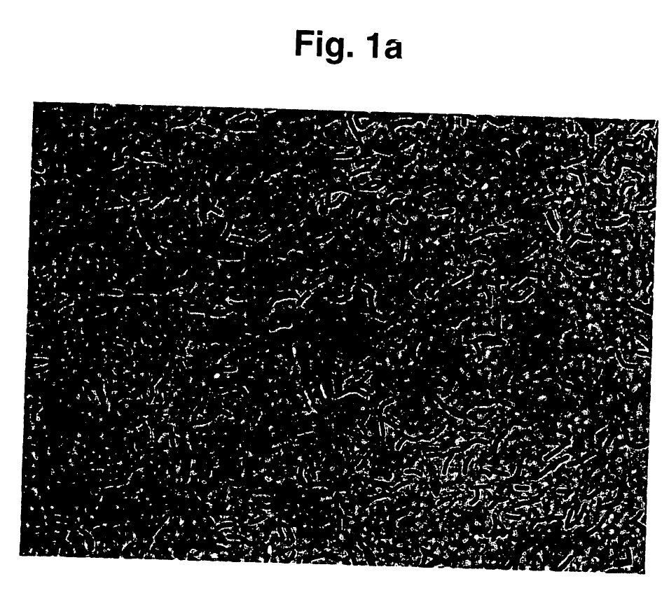

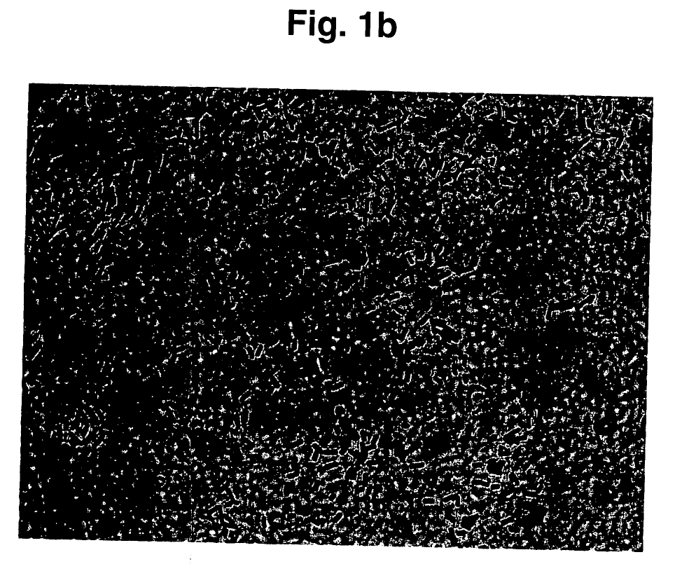

The bacterial strains of the present invention are rod-shaped Gram positive bacteria (FIGS. 1a-b), which are capable of forming endospores and producing catalase. Additional biochemical characteristics are summarized in Table 1, below.

As is illustrated in Examples 3 and 4 of the Examples section which follows, the bacterial strains of the present invention are biosafe (i.e., do not instigate systemic infections, deleterious metabolic activities, excessive immune stimulation or gene transfer) as determined using macroscopic examination of internal organs and spleen weight index evaluation.

Importantly, a co-culture of the bacterial strains of the present invention exhibits a wide range of antimicrobial activity, which is higher than that of the parental Biosporin culture (see Examples 5-8 of the Examples section which follows).

These findings suggest that the bacterial strains of the present invention would be efficacious probiotics, which can be used for treating and preventing gastrointestinal disorders in humans and animals.

According to one aspect of the present invention there is provided a biologically pure culture of bacterial strains, which exhibit a higher antagonistic activity than a Biosporin culture (see Examples 5-8 of the Examples section which follows).

According to one preferred embodiment of the present invention, the bacterial strain has all the identifying characteristics of the Bacillus subtilis HE strain, which has been deposited under the Budapest Treaty in the American Type Culture Collection (ATCC) on Jul. 8, 2003, as strain PTA-5310.

According to another preferred embodiment of the present invention, the bacterial strain has all the identifying characteristics of the Bacillus licheniformis PA strain, which has been deposited under the Budapest Treaty in the American Type Culture Collection (ATCC) on Jul. 8, 2003, as strain PTA-5311.

As used herein, the phrase “biologically pure culture” refers to a bacterial culture in which at least 20% of the bacteria are from one bacterial strain. According to preferred embodiments of this aspect of the present invention the culture is at least 30% pure, more preferably at least 40% pure, even more preferably at least 50% pure and most preferably at least 90% pure.

As mentioned hereinabove, a bacterial co-culture of the bacterial strains of the present invention (i.e. strains Bacillus lichenifonnis PA and Bacillus subtilis HE) exhibits superior antibacterial activity as compared to a Biosporin culture and therefore may be effectively used in probiotic treatment of gastrointestinal disorders.

Thus, according to yet another aspect of the present invention there is provided a bacterial co-culture including bacterial strains Bacillus licheniformis PA and Bacillus subtilis HE.

As used herein a “bacterial co-culture” refers to a bacterial cell culture, which includes at least the two bacterial strains of the present invention, described hereinabove.

It will be appreciated that the bacterial co-culture of the present invention may include other strains of probiotics bacteria, yeast (e.g., of the genus Saccharomyces, U.S. Pat. No. 6,524,575) and/or mold (e.g., of the genus Aspergillus U.S. Pat. No. 6,368,591). Examples of probiotic bacterial strains include but are not limited to the Lactobacillus genus including, but not limited to, Lactobacillus acidophilus, Lactobacillus plantarum, Lactobacillus salivarius, Lactobacillus delbrukil, Lactobacillus rhamnosus, Lactobacillus bulgaricus, Lactobacillus gaserli, Lactobacillus jensenii and Lactobacillus sporogenes; the Enterococccus genus, including Enterococcus faecium and Enterococcus thermophilus; the Bifidiobacterium genus, including Bifidobacterium longum, Bifidobacterium infantis, and Bifidobacterium bifidum; Bacillus genus, including Bacillus coagulans, Bacillus thermophilus, Bacillus laterosporus, Bacillus subtilis, Bacillus megaterium, Bacillus licheniformis, Bacillus mycoides, Bacillus pumilus, Bacillus lentus, Bacillus cereus and Bacillus circulans; Pseudomonas genus, including Pseudomonas aeruginosa, Pseudomonas putida, Pseudomonas cepacia, Pseudomonas fluorescens, and Pseudomonas 679-2; Sporolactobacillus genus; Micromonospora genus; Micrococcus genus; Rhodococcus genus and E. coli.

The isolation, identification and culturing of the bacterial strains of the present invention (i.e., Bacillus licheniformis PA and Bacillus subtilis HE) can be effected using standard microbiological techniques. Examples of such techniques may be found in Gerhardt, P. (ed.) Methods for General and Molecular Microbiology. American Society for Microbiology, Washington, D.C. (1994) and Lennette, E. H. (ed.) Manual of Clinical Microbiology, Third Edition. American Society for Microbiology, Washington, D.C. (1980).

Thus, the bacterial strains of the present invention may be derived from B. subtilis 3 and B. licheniformis 31 as described in Example 1 of the Examples section.

Isolation is preferably effected by streaking the specimen on a solid medium (e.g., nutrient agar plates) to obtain a single colony which is characterized by the phenotypic traits described hereinabove (e.g., Gram positive, capable of forming endospores aerobically) and to reduce the likelihood of working with a culture which has become contaminated and/or has accumulated mutations.

The bacterial strains of the present invention can be propagated in a liquid medium under aerobic conditions.

Medium for growing the bacterial strains of the present invention includes a carbon source, a nitrogen source and inorganic salts as well as specially required substances such as vitamins, amino acids, nucleic acids and the like.

Examples of suitable carbon sources which can be used for growing the bacterial strains of the present invention include, but are not limited to, starch, peptone, yeast extract, amino acids, sugars such as glucose, arabinose, mannose, glucosamine, maltose, and the like; salts of organic acids such as acetic acid, fumaric acid, adipic acid, propionic acid, citric acid, gluconic acid, malic acid, pyruvic acid, malonic acid and the like; alcohols such as ethanol and glycerol and the like; oil or fat such as soybean oil, rice bran oil, olive oil, corn oil, sesame oil. The amount of the carbon source added varies according to the kind of carbon source and is typically between 1 to 100 gram per liter medium. Preferably, glucose, starch, and/or peptone is contained in the medium as a major carbon source, at a concentration of 0.1-5% (W/V).

Examples of suitable nitrogen sources which can be used for growing the bacterial strains of the present invention include, but are not limited to, amino acids, yeast extract, tryptone, beef extract, peptone, potassium nitrate, ammonium nitrate, ammonium chloride, ammonium sulfate, ammonium phosphate, ammonia or combinations thereof. The amount of nitrogen source varies according the nitrogen source, typically between 0.1 to 30 gram per liter medium.

As the inorganic salts, potassium dihydrogen phosphate, dipotassium hydrogen phosphate, disodium hydrogen phosphate, magnesium sulfate, magnesium chloride, ferric sulfate, ferrous sulfate, ferric chloride, ferrous chloride, manganous sulfate, manganous chloride, zinc sulfate, zinc chloride, cupric sulfate, calcium chloride, sodium chloride, calcium carbonate, sodium carbonate can be used alone or in combination. The amount of inorganic acid varies according to the kind of the inorganic salt, typically between 0.001 to 10 gram per liter medium.

Examples of specially required substances include, but are not limited to, vitamins, nucleic acids, yeast extract, peptone, meat extract, malt extract, dried yeast and combinations thereof.

Cultivation is effected at a temperature, which allows the growth of the probiotic bacterial strains of the present invention, essentially, between 28° C. and 46° C. A preferred temperature range is 30-37° C.

For optimal growth, the medium is preferably adjusted to pH 7.0-7.4.

It will be appreciated that commercially available media may also be used to culture the bacterial strains of the present invention, such as Nutrient Broth or Nutrient Agar available from Difco, Detroit, Mich.

It will be appreciated that cultivation time may differ depending on the type of culture medium used and the concentration of sugar as a major carbon source. Typically, cultivation lasts between 24-96 hours to reach 80% sporulation of cultures.

Bacterial cells thus obtained are isolated using methods, which are well known in the art. Examples include, but are not limited to, membrane filtration and centrifugal separation.

The pH may be adjusted using sodium hydroxide and the like and the culture may be dried using a freeze dryer, until the water content becomes equal to 4% or less.

The probiotic co-culture described above, may be obtained by propagating each strain as described hereinabove. It will be appreciated that bacterial strains may be cultured together when compatible culture conditions can be employed. Alternatively, the bacterial strains of the present invention may be obtained in separate culture media for ease of standardization.

The final concentration of each bacterial strain is preferably between about 10⁹ to 10¹⁰ organisms/ml prior to combination. For enhanced antimicrobial activity the ratio between the Bacillus licheniformis PA to the Bacillus subtilis HE should be between 1:3 on a volume:volume basis. However, one of ordinary skill in the art will appreciate that this ratio may vary depending upon the culture medium used, the relative ages of the cultures and their viability.

Once a lot of the bacterial strains of the present invention is generated, it is preferably quality qualified. Such qualification may include testing resistance to gastric acidity, resistance to bile acid, which correlates with gastric survival in vivo, adherence to mucus and/or human epithelial cells and cell lines, antimicrobial activity against potentially pathogenic bacteria, ability to reduce pathogen adhesion to surfaces and bile salt hydrolase activity [Conway (1987) J. Dairy Sci. 70:1-12].

The wide-ranged and high antibacterial activities (see Examples 5-8 of the Examples section) of the bacterial strains of the present invention suggest usage thereof in treating or preventing a variety of gastrointestinal disorders.

Thus, according to still another aspect of the present invention there is provided a method of treating or preventing a gastrointestinal disorder in a subject.

The method is effected by administering to a subject in need thereof a therapeutically effective amount of the probiotic bacterial strains of the present invention. It will be appreciated that besides viable cells, non-viable cells such as killed cultures or compositions containing beneficial factors expressed by the probiotic bacteria of the present invention can also be administered. This could include thermally killed cells or bacterial cells killed by exposure to altered pH or subjection to pressure. It will be appreciated that compositions including non-viable bacterial products are simpler to generate and store.

As used herein the term “treating” refers to alleviating or diminishing a symptom associated with a gastrointestinal disorder. Preferably, treating cures, e.g., substantially eliminates, the symptoms associated with the gastrointestinal disorder.

Subjects which may be treated with the bacterial cultures of the present invention include humans and animals which may benefit from probiotic treatment. Examples include but are not limited to mammals, reptiles, birds, fish and the like.

Examples of gastrointestinal disorders which may be treated using the probiotic strains of the present invention include, but are not limited to, acute diarrhea, traveller's diarrhea, lactose intolerance, HIV-associated diarrhea, sucrose isomaltase deficiency, inflammatory bowel disease, pouchitis, carcinogenesis, enteral feeding associated diarrhea, antibiotic associated diarrhea, small bowel bacterial overgrowth, irritable bowel syndrome and disorders which are associated with enteropathogens such as Helicobacter pylori, Campylobacter jejuni, Campylobacter coli, Staphylococcus aureus, Staphylococcus epidermidis, Streptococcus pyogenes, Streptococcus pneumoniae, Enterococcus faecalis, Haemophilus influenzae, Escherichia coli, Klebsiella pneumoniae, Enterobacter cloacae, Citrobacter freundii, Serratia marcescens, Pseudomonas aeruginosa and Pseudomonas maltophilia, Salmonella sp. Viruses such as rotavirus and fungi such as Candida albicans and Aspergillus fumigatus, and combinations of these species (see Background section).

“Merck's Veterinary Manual” provides a detailed description of animal's gastrointestinal disorders, which can be treated according to this aspect of the present invention. Examples include but are not limited to diseases-associated with pathogens of horses including horse bots, lip bots or throat bots, caused by Gasterophilus species, such as G. intestinalis, G. haemorrhiodalis, and G. nasalis, stomach worms, caused by Habronema species, such as H. muscae or H. microstoma mulus, or caused by Crascia species, such as C. mepastoma, or caused by Trichostrongvlus species, such as T. axei, ascarids (white worms) caused by Parascaris species such as P. eciuorum, blood worms (palisade worms, red worms or sclerostomes) caused by Stroncrvlus species such as S. vulcraris, S. epuinus or S. edentatus, small strongyles of the cecum and colon caused by Triodontophorus species such as T. tenuicollis, pinworms caused by Oxvuris species such as O. eaui, strongyloides infections of the intestine caused by Stroncivloides westeri, tapeworms caused by Anonlocephala species such as A. macma and A. perfoliata, and caused by Paranonlocephala mamillana.

Various other pathogens cause disease in ruminants, typically cattle, including the wire worm (or barber's pole worm or large stomach worm) caused by Haemonchus species. Pathogens caused in non-ruminants, typically swine, include stomach worms caused by Hvostroncmulus species.

Additional pathogens are known to infect a variety of animal hosts, and therefore are a target for treatment by the methods of the present invention. For example, gastrointestinal pathogens infect a variety of animals and can include Spirocerca species such as S. lupi which cause esophageal worms in canines and Physoloptera species that cause stomach worms in canines and felines.

It will be appreciated that the bacterial strains of the present invention may be used to treat other diseases or disorders (i.e., extraintestinal), which may be treated by probiotics.

The ability of the bacterial strains of the present invention to treat bacterial, fungal or viral infections in other organs is an outcome of stimulating multiple defense mechanisms [reviewed by Isolauri (2001) Am. J. Clin. Nut. 73:444S-450S] including promotion of a nonimmunologic gut defense barrier which may inhibit translocation of potential pathogens and thus prevent infections of the blood stream and other tissues or organs. Another defense mechanism is improvement of the intestine's immunologic barrier, particularly through intestinal immunoglobulins A responses and alleviation of intestinal inflammatory responses which produce a gut stabilizing effect. As well as by immune regulation, particularly through balance control of proinflammatory and anti-inflammatory cytokines.

Examples of extraintestinal diseases which can be treated with the probiotic cultures of the present invention include, but are not limited to appendicitis, autoimmune disorders, multiple sclerosis, Alzheimer's disease, rheumatoid arthritis, coeliac disease, diabetes mellitus, organ transplantation, periodontal disease, urogenital diseases (vaginal, urethral and perineal), sexually transmitted disease, HIV infection, HIV replication, surgical associated trauma, surgical-induced metastatic disease, sepsis, weight loss, anorexia, fever control, cachexia, wound healing, ulcers, gut barrier function, allergy, asthma, respiratory disorders, rhinovirus-associated diseases (e.g., otitis media, sinusitis, asthma and pulmonary diseases), circulatory disorders, coronary heart disease, anaemia, disorders of the blood coagulation system, renal disease, disorders of the central nervous system, hepatic diseases (e.g., hepatic encephalopathy) constipation, ischaemia, nutritional disorders, osteoporosis, endocrine disorders, epidermal disorders, psoriasis, anthrax and/or acne vulgaris [see Examples 5-8, U.S. patent application No. 20030113306, Rolfe (2000) Journal of Nutrition 130:396S-402S and Background section].

Typical concentration range of probiotic microorganisms administered, according to this aspect of the present invention, is 10³ to 10¹³ cells per day. Preferably, at least about 10⁶, at least about 10⁷, at least about 10⁸ cells per day are used in probiotic administration (see U.S. Pat. Nos. 6,221,350 and 6,410,305). However, it will be appreciated that the amount of bacteria to be administered will vary according to a number of parameters including subject's size, type of disorder and severity of symptoms.

The bacterial cultures of the present invention can be formulated in a nutritional composition (e.g., foodstuff, food additive or feed additive). For example, the bacterial strains of the present invention may be included in fermented milk products (i.e., nutraceuticlas), such as described in U.S. Pat. No. 6,156,320.

Alternatively, the bacterial strains of the present invention may be formulated in a pharmaceutical composition, where it is mixed with a pharmaceutically acceptable carrier for any type of administration route, selected according to the intended use.

Herein the term “active ingredient” refers to the bacterial preparation accountable for the biological effect.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

In addition to carriers the pharmaceutical compositions or nutritional compositions of the present invention may also include, colonization carriers, nutrients, antibiotics, anti-fungal agents, antioxidants, plant extracts, buffering agents, coloring agents, flavorings, vitamins and minerals, which are selected according to the intended use and the route of administration employed.

Colonization carriers—The compositions of the present invention may include a colonization carrier which transports the probiotic microorganisms to the large bowel or other regions of the gastrointestinal tract. Typically the carrier is a saccharide such as amylose, inulin, pectin, guar gum, chitosan, dextrans, cyclodextrins and chondroitin sulphate [Chourasia and Jain (2003) J. Pharm. Pharmaceut. Sci. 6:33-66].

Preferably, modified and/or unmodified resistant starches are used as colonization carriers (see U.S. Pat. No. 6,221,350).

The phrase “resistant starch” refers to starch forms defined as RS1, RS2, RS3 and RS4 as defined in Brown, McNaught and Moloney (1995) Food Australia 47: 272-275. Typically, a resistant starch is used in a probiotic composition since it is essentially not degraded until it reaches the large bowel. Therefore it provides a readily available substrate for fermentation by the probiotic microorganisms once they reach the large bowel. Preferably, the resistant starch is a high amylose starch, including but not limited to maize starch having an amylose content of 50% w/w or more, particularly 80% w/w or more, rice and wheat starch having an amylose content of 27% w/w or more and; particular granular size ranges of starches having an amylose content of 50% or more and enhanced resistant starch content, these starches including maize, barley, wheat and legumes. Other forms of resistant starch derived from sources such as bananas or other fruit types, tubers such as potatoes, and mixtures or combinations thereof can also be used in accordance with the present invention.

It will be appreciated that it may be advantageous to chemically modify the starch, such as by altering the charge, density or hydrophobicity of the granule and/or granule surface to enhance the attachment compatibility between the microorganism and the resistant starch. Chemical modifications, such as etherification, esterification, acidification and the like are well known in the art and may be utilized to modify the starch. Alternatively, modifications can be induced physically or enzymically such as described in U.S. Pat. No. 6,221,350.

The colonizing carrier may also be an oligosaccharide. Oligosaccharides are known to increase the number of probiotic microorganisms in the gastrointestinal tract. Examples of commercially available oligosaccharides which can be used as colonizing carriers include but are not limited fructo-, galacto-, malto-, isomalto-, gentio-, xylo-, palatinose-, soybean- (including raffinose and stachyose), chito-, agaro-, neoagaro-, gluco-, β-gluco-, cyclo-inulo-, glycosylsucrose, lactulose, lactosucrose and xylsucrose.

The oligosaccharide can be used in the composition in a concentration of about 0.01 to 10% (w/w). Preferably the concentration of the oligosaccharide is about 0.05 to 5%.

Preferably, a combination of starch and an oligosaccharide is used as the colonizing agent of this aspect of the present invention.

Antibiotics—The compositions of the present invention may include a therapeutically-effective amount of a preferably, broad-spectrum antibiotic. Measures are taken to include an antibiotic or a concentration thereof, which does not affect the bacterial strains of the present invention (see Table 4, below). For example the bacterial strains of the present invention may be combined with a therapeutic dose of an antibiotic such as Cefuroxime of the Cephalosporin antibiotic family. However, other antibiotics can also be used according to this aspect of the present invention [Fursikova T. M., Sorokulova I. B., Sergiychuk M. G., Sichkar S. V., Smirnov V. V. (2000) The effect of antibiotics and their combination with probiotics on mice intestine microflora, Microbiologichny Zhurnal, 62, N3, 26-35].

A therapeutic composition of the present invention may contain approximately 1 to 250 mg of the selected antibiotic per unit of composition.

Anti-fungal agents—The compositions of the present invention may include a therapeutically-effective amount of an anti-fungal agent. Typical anti-fungal agents which may be utilized include, but are not limited to: Clotrimazole, Fluconazole, Itraconazole, Ketoconazole, Miconazole, Nystatin, Terbinafine, Terconazole, Tioconazole, and the like.

Antioxidants, buffering agents, plant extracts, coloring agents, flavorings, vitamins and minerals—The compositions of the present invention may include antioxidants, buffering agents, plant extracts and other agents such as coloring agents, flavorings, vitamins or minerals. For example, the composition of the present invention may contain one or more of the following minerals: calcium citrate (15-350 mg); potassium gluconate (5-150 mg); magnesium citrate (5-15 mg); and chromium picollinate (5-200 μg). In addition, a variety of salts may be utilized, including calcium citrate, potassium gluconate, magnesium citrate and chromium picollinate. Chemicals are commercially available from Spectrum Quality Products, Inc (Gardena, Calif.), Sigma Chemicals (St. Louis, Mo.), Seltzer Chemicals, Inc., (Carlsbad, Calif.) and Jarchem Industries, Inc., (Newark, N.J.). Examples of plant extracts, which can be used in accordance with the present invention include but are not limited to chamomile, bur-marigold, St. John's wort, ginger and other approved plant extracts which are FDA approved [for review see O'Hara M, Kiefer D, Farrell K, Kemper K. Arch Fam Med. (1998) November-December;7(6):523-36.; Modesto A, Lima K C, de Uzeda M. ASDC J Dent Child. (2000) September-October;67(5):338-44,302; Lee K G, Shibamoto T. J Agric Food Chem. Aug. 14 (2002);50(17):4947-52].

Thickening agents—Thickening agents may be added to the compositions such as polyvinylpyrrolidone, polyethylene glycol or carboxymethylcellulose.

Carriers—The active agents (e.g., bacterial cells) of the compositions of the present invention are combined with a carrier, which is physiologically compatible with the tissue of the species to which it is administered (i.e., suitable for human consumption or animal consumption). The carriers, according to this aspect of the present invention can be solid-based, dry materials for formulation into tablet, capsule or powdered form. Alternatively, the carrier can be of liquid or gel-based materials for formulations into liquid or gel forms. The specific type of carrier, as well as the final formulation depends, in part, upon the selected route(s) of administration.

Typical carriers for dry formulations include, but are not limited to: trehalose, malto-dextrin, rice flour, micro-crystalline cellulose (MCC), magnesium stearate, inositol, fructo-oligosaccharides (FOS), gluco-oligosaccharide (GOS), dextrose, sucrose, and the like. Where the composition is dry and includes evaporated oils that may cause the composition to cake (i.e., adherence of the component spores, salts, powders and oils), it is preferred to include dry fillers, which distribute the components and prevent caking. Exemplary anti-caking agents include MCC, talc, diatomaceous earth, amorphous silica, gelatin, saccharose, skimmed dry milk powder, starch and the like, which are typically added in an amount of from approximately 1% to 95% by weight. It will be appreciated that dry formulations, which are subsequently rehydrated (e.g., liquid formula) or given in the dry state (e.g., chewable wafers, pellets or tablets) are preferred to initially hydrated formulations. Dry formulations (e.g., powders) may be added to supplement commercially available foods (e.g., liquid formulas, strained foods, or drinking water supplies).

Suitable liquid or gel-based carriers include but are not limited to: water and physiological salt solutions; urea; alcohols and derivatives (e.g., methanol, ethanol, propanol, butanol); glycols (e.g., ethylene glycol, propylene glycol, and the like). Preferably, water-based carriers have a neutral pH value (i.e., pH 7.0).

Preservatives may also be included within the carrier including methylparaben, propylparaben, benzyl alcohol and ethylene diamine tetraacetate salts. The compositions of the present invention may also include a plasticizer such as glycerol or polyethylene glycol (with a preferred molecular weight of MW=800 to 20,000). The composition of the carrier can be varied so long as it does not interfere significantly with the pharmacological activity of the active ingredients or the viability of the bacterial strains of the present invention. Other types of carriers, which can be used according to this aspect of the present invention are described hereinbelow.

Spore germination inhibitor—When liquid-based compositions containing spores are provided, it is desirable to include a spore germination inhibitor to promote long term storage. Any spore germination inhibitor may be used. Preferred inhibitors include: hyper-saline carriers, methylparaben, guar gum, polysorbates, preservatives, and the like.

Nutrient supplements—A nutrient supplement component of the compositions of the present invention can include any of a variety of nutritional agents, which are well known in the art, including vitamins, minerals, essential and non-essential amino acids, carbohydrates, lipids, foodstuffs, dietary supplements, and the like. Thus, the compositions of the present invention can include fiber, enzymes and other nutrients. Preferred fibers include, but are not limited to: psyllium, rice bran, oat bran, corn bran, wheat bran, fruit fiber and the like. Dietary or supplementary enzymes such as lactase, amylase, glucanase, catalase and the like can also be included. Vitamins for use in the compositions of the present invention include vitamins B, C, D, E, folic acid, K, niacin, and the like. Typical vitamins are those, recommended for daily consumption and in the recommended daily amount (RDA).

The pharmaceutical composition of the present invention is formulated according to the intended use. A review of conventional formulation techniques can be found in e.g. “The Theory and Practice of Industrial Pharmacy” (Ed. Lachman L. et al, 1986) or Laulund (1994).

In any case, suitable routes of administration may, for example, include topical, intravaginal, trans-urethral, oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

For injection, the active ingredients of the invention may be formulated in aqueous solutions, preferably in physiologically compatible buffers such as Hank's solution, Ringer's solution, or physiological salt buffer.

For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

It will be appreciated that the compositions of the present invention can be encapsulated into an enterically-coated, time-released capsule or tablet. The enteric coating allows the capsule/tablet to remain intact (i.e., undissolved) as it passes through the gastrointestinal tract, until such time as it reaches the small intestine.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount (see U.S. Pat. No. 6,448,224). Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion.

A number of examples for parenteral administration of live bacteria cells are known in the art [see for example, Tjuvajev (2001) J. Control Release 74(1-3):313-5. Rosenberg (2002) J. Immunother. 25:218-25; Sheil (2004) Gut 53(5):694-700; and Matsuzaki (2000) Immunol. Cell Biol. 78(1):67-73]. It will be appreciated that bacteria cells of the present invention may also be administered in an attenuated form so as to modulate immune responses [Matsuzaki (2000) Immunol. Cell Biol. 78(1):67-73].

Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in a powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Formulations suitable for genital application include cream, ointment, lotion, jelly, solution, emulsion, spray or foam formulation.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Formulations suitable for vaginal administration may be presented as pessaries, tampons, creams, gels, pastes, jelly, foams or sprays or aqueous or oily suspensions, solutions or emulsions (i.e., liquid formulations), or films containing carriers as are known in the art to be appropriate (described in details in U.S. Pat. No. 5,756,681).

Compositions suitable for application to the vagina are disclosed in U.S. Pat. Nos.: 2,149,240, 2,330,846, 2,436,184, 2,467,884, 2,541,103, 2,623,839, 2,623,841, 3,062,715, 3,067,743, 3,108,043, 3,174,900, 3,244,589, 4,093,730, 4,187,286, 4,283,325, 4,321,277, 4,368,186, 4,371,518, 4,389,330, 4,415,585, 4,551,148, 4,999,342, 5,013,544, 5,227,160, 5,229,423, 5,314,917, 5,380,523, and 5,387,611.

For transurethral administration the composition contains one or more selected carriers excipients, such as water, silicone, waxes, petroleum jelly, polyethylene glycol (PEG), propylene glycol (PG), liposomes, sugars such as mannitol and lactose, and/or a variety of other materials, with polyethylene glycol and derivatives thereof. It is preferred that the pharmaceutical compositions contain one or more transurethral permeation enhancers, i.e., compounds which act to increase the rate at which the selected drug permeates through the urethral membrane. Examples of suitable permeation enhancers include dimethylsulfoxide (DMSO), dimethyl formamide (DMF), N,N-dimethylacetamide (DMA), decylmethylsulfoxide, polyethylene glycol monolaurate (PEGML), glycerol monolaurate, lecithin, the 1-substituted azacycloheptan-2-ones, particularly 1-n-dodecylcyclaza-cycloheptan-2-one (available under the trademark Azone^{RTM} from Nelson Research & Development Co., Irvine, Calif.), SEPA^{RTM} (available from Macrochem Co., Lexington, Mass.), alcohols (e.g., ethanol), surfactants including, for example, Tergitol^{RTM}, Nonoxynol-9^{RTM} and TWEEN-80^{RTM}, and lower alkanols such as ethanol. As disclosed in WO91/16021, transurethral administration of an agent can be carried out in a number of different ways. For example, the agent can be introduced into the urethra from a flexible tube, squeeze bottle, pump or aerosol spray. The agent may also be contained in coatings, pellets or suppositories, which are absorbed, melted or bioeroded in the urethra. In certain embodiments, the agent is included in a coating on the exterior surface of a penile insert.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

Typically bacteria species of the present invention (i.e., active ingredient) may constitute 1-90%, more preferably 5-90%, even more preferably 10-90% by weight of the final composition and still more preferably 15-88% % by weight contained within a formulation suitable for administration. Alternatively, the composition of the present invention may contain at least 10⁶, more preferably at least 10⁸, even more preferably at least 10¹⁰ viable bacteria per one dose of composition.

Toxicity and therapeutic efficacy of the active ingredients described herein can be determined by standard pharmaceutical procedures in vitro, in cell cultures or experimental animals (see Examples 1-4 of the Examples section which follows). The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

As mentioned hereinabove, the bacterial strains of the present invention can be included in the compositions of the present invention in a sporolated form. Methods of generating spores are well known in the art. Alternatively, the bacterial strains can be included in the composition in the form of a lyophilized (dried) cell mass.

Spores may be incorporated into any type of dry or lyophilized product, which is dissolved or mixed for example with hot water since of spores display resistance to a high temperature (e.g., 90° C. for 10 min). It will be appreciated that the bacterial spores may either be incorporated into the dry or lyophilized product by the manufacturer of the product or by the consumer during preparation. These dry or lyophilized products include, but are not limited to: tea bags, coffee (e.g., “freeze-dried” or ground), sweeteners (e.g., synthetic (NutraSweet^{RTM}) and natural); hot cereal (e.g., oatmeal, Cream of Wheat^{RTM}, and the like), hot beverage condiments/flavorings and creamers, and the like.

Alternatively, spores may be utilized as a dry or lyophilized product, or incorporated into a chewable tablet, toothpaste, mouthwash, oral drops, and the like in order to inhibit the formation of dental caries, gingivitis, and other forms of periodontal disease or oral infections caused by yeast, Herpes simplex I (, and various other infections caused by oral pathogens.

It will be appreciated that the bacterial cells or spores may be incorporated into an aqueous solution (e.g., physiological saline) to directly administer the probiotic bacteria to the colon (via an enema or the like).

As mentioned hereinabove, the probiotic compositions of the present invention can be provided to animals using methods, which are well known in the art.

Typically, the probiotic composition is introduced into the animal's gastrointestinal tract via a feed additive, which is added to a feed diet. Alternative methods of administration are liquid ingestion, paste or gel ingestion, boles, powder dusting surface of animal and the like.

In addition to probiotic bacterial cells, the feed additive may include, for example, carrier materials such as, limestone and wheat midds (see U.S. Pat. No. 6,410,305). The feed additive can be added to the animal's regular diet at a rate of 0.01 to 10 and preferably about 0.5 to 2.5 pounds of additive per ton of animal feed;

The feed additive may contain about 0.3% to about 20% by weight of probiotic bacterial cells. Preferably the feed additive contains 7% to 15% by weight probiotic premix and most preferably about 10% to 13% by weight.

It will be further appreciated that the probiotic microorganisms of the present invention do not adhere to the intestinal epithelium. Thus in the absence of a repeat dosage, the bacteria remain in the gastrointestinal tract for maximal time of approximately 3-5 days and are considered to be a transient flora. The relatively rapid gastrointestinal-clearance time and inability to adhere to the gastrointestinal epithelium of Bacillus coagulans, has the advantage of preventing the later development of bacteremia in, for example, immunocompromised individuals.

The bacterial strains and or compositions of the present invention can be included in a product identified for treating a particular disorder such as described above. Typically, the product is in the form of a package containing the bacterial cells or compositions including same, or in combination with packaging material. The packaging material is selected to retain bacterial viability and includes a label or instructions for, for example, use of the components of the package. The instructions indicate the contemplated use of the packaged component, as described herein for the methods or compositions of the invention, contents (e.g., genus, species, strain designation), minimum numbers of viable bacteria at end of shelf-life, proper storage conditions and corporate contact details for consumer information. The label may also provide information related to the freshness of the product. This information may include a date of manufacture, a “sell be” date or a “best before date”. A “sell by” date specifies by which date the product should have been sold to the consumer. A “best before” date specifies by when the product should be disposed of by vendor or consumer. Alternatively or additionally “active labeling” may be used. For example, U.S. Pat. Nos. 4,292,916, 5,053,339 5,446,705 and 5,633,835 describe color changing devices for monitoring the shelf-life of perishable products. These devices are initiated by physically bringing into contact reactive layers so that the reaction will start, and this action can only conveniently be performed at the time of packaging. This approach is suitable for monitoring the degradation of foodstuffs which lose freshness throughout the entire distribution chain. U.S. Pat. No. 5,555,223 describes a process for attaching timing indicators to packaging, including the step of setting the timer clock at the exact time of production.

Depending upon the intended use, the product may optionally contain either combined or in separate packages one or more of the following components: colonization carriers, flavorings, carriers, and the like components. For example, the product can include spores for use in combination with a conventional liquid product, together with instructions for combining the probiotic with the formula for use in a therapeutic method.

The bacterial strains of the present invention can also be used as pharmaceutical delivery systems. It will be appreciated that such delivery systems are inherently safer than the use of attenuated pathogens in humans, including infants, the elderly and individuals whose immune function is impaired [Grangette (2001) Infect. Immun. 69:1547-1553].

The bacterial strains of the present invention can also be modified to express heterologous expression products using expression systems, which are well known in the art. This approach was used to reduce colitis in mice intragastrically administered with the IL-10-secreting L. lactis strain [Steidler (2000) Science 289:1352-1355].

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Isolation of Probiotic Strains Bacillus subtilis HE and Bacillus licheniformis PA

The probiotic organisms Bacillus subtilis HE and Bacillus licheniformis PA were isolated from B. subtilis 3 and B. licheniformis 31 (i.e., Biosporin) together by sequential transfer on milk agar and selection for lysozyme production.

Experimental Procedures

Selection of clones in activity of decomposition of casein—Milk agar was produced by preparing a milk solution including 5 g skim milk powder (Medallion Milk Co Ltd, 10-59 Scurfield Blvd, Winnipeg, Manitoba, Canada; The Carbery Group, Ballineen Co. Cork, Ireland) in 50 ml distilled water; and an agar solution including 1 g agar (Oxoid, Laboratory Preparations) in 50 ml distilled water. Each solution was autoclaved at 121° C. for 20 min, and left to cool to 45° C. The autoclaved solutions were mixed together and poured into Petri dishes (Falcon). Bacteria were grown on agar milk as isolated colonies. Colonies with high casein decomposing activity were selected for another transfer on milk agar. Procedure was repeated 5 times, after which selected clones were selected for lysozyme production.

Selection of clones producing lysozyme—Each bacterial culture was plated on Petri dishes with nutrient agar [Difco, Detroit, Mich.] and cultivated at 37° C. for 24 h to obtain isolated colonies. Each colony was transferred to another plate and bacteria on parental plates were killed by placing the plates in a bell jar and exposing thereof to an atmosphere of saturated chloroform vapor for 20 min [Harasawa et al. (1980) Antimicrob. Agents Chemother. 18: 58-62]. Plates with viable bacteria were then covered with a thin layer of 0.8% nutrient agar including Micrococcus luteus CCM 1423 (10⁶ CFU/ml) and incubated at 37° C. for 24-48 h. Zones of Micrococcus luteus growth inhibition were observed and clones exhibiting high lysozyme production were selected for a next round of selection. Altogether, seven rounds of selection were effected.

### Example 2

### Characterization of Probiotic Strains Bacillus subtilis HE and Bacillus licheniformis PA

Materials and Experimental Procedures

Endospore formation—Bacterial strains were grown on nutrient agar containing manganese (Beef extract, 3 g; peptone, 5 g; hydrous manganese sulfate, 5 mg; agar, 15 g; distilled water, 1000 ml; pH 6.8) at 37° C. for 24-72 h. A turbid suspension of bacteria in saline was placed on slide and covered with glass. Spores were observed using a phase-contrast microscope.

Catalase activity—Bacterial cultures grown for 1 or 2 days on slants of nutrient agar were flooded with 0.5 ml of 10% hydrogen peroxide and gas production was determined as previously described [Sneath, P. H. A (1986) Endospore-forming gram-positive rods and cocci. In: Sneath, P. H. A., Mair, N. S., Sharpe, M. E., and Holt, J. G. (ed.), Bergey's Manual of Systematic Bacteriology, vol. 2. Lippincott Williams & Wilkins, Baltimore, pp.1104-1207].

Egg-yolk broth preparation and lecithinase production—Basal medium including Tryptone, 10 g; disodium hydrogen phosphate, 5 g; potassium dihydrogen phosphate, 1 g; sodium chloride, 2 g; magnesium sulfate 7H₂O, 0.1 g; glucose, 2 g; distilled water, 1000 ml, pH 7.6 was prepared, autoclaved at 121° C. and cooled for 20 min. 1.5 ml egg-yolk aspirated aseptically (or a sterile commercial preparation thereof used according to manufacturer's instructions), was added to 100 ml of basal medium. The medium was allowed to stand overnight at 4° C. Supernatant was dispensed in sterile tubes in 2.5-ml aliquots. Basal medium without added egg yolk was similarly dispensed. Bacteria were inoculated into tubes of control broth and yolk broth. The appearance of a heavy white precipitate in or on the surface of the egg-yolk containing medium was observed following incubation at 37° C. for 1, 3, 5 and 7 days.

Anaerobic growth—Bacterial cultures were inoculated into tubes with 75-mm deep anaerobic agar (Trypticase, 20 g; glucose, 10 g; sodium chloride, 5 g; agar, 15 g; sodium thioglycolate, 2 g; sodium formaldehyde sulfoxylate, 1 g; distilled water, 1 liter, pH 7.2.) with a small (outside diameter 1.5 mm) loopful of nutrient broth culture, which was formed by stubbing the bottom of the culture tube. Bacteria were incubated at 37° C. and growth was recorded at days 3 and 7.

Nitrogen production—Bacterial cultures were grown in nitrate broth including peptone, 5 g; beef extract, 3 g; potassium nitrate, 1 g; distilled water, 1000 ml; pH 7.0. The medium was poured into test tubes containing invert Durham's tubes and sterilized by autoclaving at 121° C. for 20 min. Accumulation of nitrogen was observed following 3 of 7 days of incubation at 37° C.

Propionate utilization—Bacterial cultures were inoculated onto the slants of propionate utilization medium (Sodium propionate, 2 g; magnesium sulfate 7H₂O, 1.2 g; diammonium hydrogen phosphate, 0.5 g; potassium chloride, 1 g; trace element solution (see below), 40 ml; agar, 15 g; distilled water, 920 ml; 0.04% (w/v) solution of phenol red, 20 ml, pH 6.8) and incubated for 14 days at 37° C. The development of red color was indicative of propionate utilization.

Production of hemolysins—Bacterial cultures were inoculated onto the sheep blood agar (see below) and incubated for 24-72 h at 37° C. Hemolysin production was detected by the formation of clear zones surrounding the bacterial colonies resulting from hemolytic activity. Note that clear zones were absent when hemolytic activity was absent.

Sheep blood agar—5% sterile defibrinated sheep blood was aseptically added to Blood Agar Base (Difco) prepared according manufacturer instruction and cooled to 45-50° C. The solution was mixed well and dispensed into sterile Petri dishes.

Production of arginine dihydrolase—A tube with the Sherris medium and control tube (i.e., without arginine) were inoculated with overnight bacterial culture to which sterile vasoline oil was added. The tubes were incubated at 37° C. for 5 days. Production of arginine dihydrolase was detected by the appearance of violet colour in the medium with arginine.

Sherris medium—Peptone, 1 g; beef extract, 5 g; pyridoxine, 0.005 g; glucose, 0.5 g; L-arginine monohydrochloride, 10 g; bromcresol purpur, 0.01 g; cresol rot, 0.005 g; distilled water, 1000 ml.

Poly-β-hydroxybutyrate synthesis—Bacterial cultures were inoculated onto nutrient agar supplemented with 1% glucose. Following incubation at 37° C. for 24 h slides with the cultures were prepared, stained with crystal violet and studied microscopically. Note that the globules of poly-β-hydroxybutyrate were observed as non stained particles.

Results

Bacterial classification tests found that Bacillus subtilis HE and Bacillus licheniformis PA are rod-shaped Gram-positive bacteria (FIGS. 1a-b), which are capable of forming endospores and producing catalase. Neither strain is capable of forming poly-β-hydroxybutyrate, producing egg-yolk lecithinase nor hemolysins.

The strains exhibited different biochemical characteristics. As summarized in Table 1, below, strain Bacillus licheniformis PA, as opposed to strain Bacillus subtilis HE, was capable of growing under anaerobic conditions, producing arginine dihydrolase, forming gas from nitrate and utilizing propionate.

Altogether, these characteristics confirmed that strain Bacillus subtilis HE was B. subtilis and strain Bacillus licheniformis PA was B. licheniformis. The 16S rDNAs of strain Bacillus subtilis HE and strain Bacillus licheniformis PA are as set forth is SEQ ID NOs: 1 and 2, respectively.

TABLE 1

| Characteristics | B. subtilis/HE | B. licheniformis/PA |
|---|---|---|
| Cell diameter > 1.0 μm | — | — |
| Spores round | — | — |
| Sporangium swollen | — | — |
| Catalase | + | + |
| Anaerobic growth | — | + |
| Voges-Proskauer test | + | + |
| Acid from | | |
| Glucose | + | + |
| Arabinose | + | + |
| Xylose | + | — |
| Mannitol | + | + |
| Utilization of | | |
| Citrate | + | + |
| Propionate | — | + |
| Hydrolysis of | | |
| Starch | + | + |
| Urea | — | — |
| Nitrate reduced to nitrite | + | + |
| Gas from nitrate | — | + |
| Reduction of methylene blue | + | + |
| Arginine dehydrolase | — | + |
| Egg-yolk lecithinase | — | — |
| Hemolysis | — | — |
| Formation of poly-β- | — | — |
| hydroxybutyrate | | |

### Example 3

### Acute and Chronic Toxic Effects of Probiotic Strains Bacillus subtilis HE and Bacillus licheniformis PA

Experimental Procedures

Acute toxicity—Mice were acclimatised under experimental conditions for 7 days, following which they were randomly assigned to 21 different groups of 10 mice each. Bacterial cultures were administrated intravenously and intraperitoneally at the different levels of 5×10⁷, 5×10⁸, 5×10⁹ CFU/mouse and orally at 5×10⁷, 5×10⁸, 2×10¹¹ CFU/mouse. Mice of the control group were given sterile saline. Animals Were observed for 7 days. During this period, activity, behaviour and hair lustre of each mouse were recorded daily. After 1 and 7 days, five animals from each group were euthanasied by ether overdose and internal organs were observed macroscopically. Samples of different organs and tissues including liver, kidneys, lungs, spleen, intestine, mesenteric lymph nodes, brain, thymus and tissues around the throat (for the groups, treated orally) were collected for histological analysis:

Chronic toxicity study—The chronic toxicity study was carried out using mice and rabbits. Ten animals of each species (for each bacterial strain) were orally inoculated with bacterial cultures at doses of: mice, 1×10⁶ CFU/day; rabbits, 1×10⁹ CFU/day. Ten animals of each species in the control group received sterile saline. The treatment lasted 10 days, during which the activity and behaviour of each animal were observed. On day 11, all animals were euthanasied humanely and internal organs were observed macroscopically. Samples of different organs and tissues were collected for histological analysis: liver, kidneys, lungs, spleen, intestine, mesenteric lymph nodes, brain, thymus and tissues around throat.

In additional experiments, 20 rabbits (ten for each bacterial strain) were orally inoculated with bacterial cultures at a dose of 1×10⁹ CFU/day for 30 days. Ten control rabbits received sterile saline. On day 31, all animals were euthanasied humanely and samples of blood and different organs and tissues were collected.

Results

During the entire experimental period, there was no noticeable change in activity and behavior in any of the animals. All animals were clinically healthy, i.e. no diarrhea or other treatment-related illness or death was recorded.

There were no differences in the appearance of visceral organs between experimental and control groups of animals as determined under macroscopic examination. Furthermore, there was no significant difference in spleen weight index (SWI) of control and orally inoculated mice, as shown in Table 2, below.

TABLE 2

| Groups of mice | Number of mice in group | SWI^{a} |
|---|---|---|
| Bacillus subtilis HE | 10 | 3.41 ± 0.18 |
| B. licheniformis PA | 10 | 3.37 ± 0.16 |
| Control | 10 | 3.40 ± 0.14 |

SWI = spleen weight (mg)/mouse body weight (g)

Microscopic observation found no signs of pathology in all analyzed organs and tissues both during the study of acute toxicity in mice and chronic toxicity in mice and rabbits.

No pathology was observed in any of the organs and tissues of mice and of mice and rabbits, during the acute and chronic toxicity study, respectively.

Furthermore, as shown in Table 3, below, there were no differences in the hematological index measured in blood of control and treated rabbits orally administered with the bacterial strains of the present invention for 30 days.

TABLE 3

| | Control | B. subtilis HE | B. licheniformis PA |
|---|---|---|---|
| Sedimentation rate | 1-2 | 1-2 | 1-2 |
| (mm/h) | | | |
| Hemoglobin (g/l) | 123.80 ± 6.20 | 130.33 ± 7.30 | 127.50 ± 6.80 |
| RBC count (×10¹²⁾ | 5.30 ± 1.80 | 5.60 ± 1.50 | 5.50 ± 1.40 |
| Leukocytes count | 7.80 ± 0.80 | 7.40 ± 0.60 | 7.20 ± 0.90 |
| (×10⁹⁾ | | | |
| Neutrophils (%) | 43.27 ± 3.70 | 43.52 ± 3.69 | 42.62 ± 3.39 |
| Lymphocytes (%) | 49.40 ± 2.07 | 48.90 ± 2.91 | 49.89 ± 1.26 |
| Monocytes (%) | 3.60 ± 0.90 | 3.39 ± 0.80 | 3.62 ± 0.90 |
| Eosinophils (%) | 3.73 ± 1.30 | 4.19 ± 1.20 | 3.87 ± 1.30 |

### Example 4

### Antibiotic Resistance of the Probiotic Strains Bacillus subtilis HE and Bacillus licheniformis PA

Antibiograms for strains were obtained by the disc diffusion method according to the recommendations of the National Committee for Clinical Laboratory. Standards (1997). Overnight broth cultures of tested strains after growth in LB (including Bacto tryptone, 10 g; Bacto yeast extract, 5 g; sodium chloride, 5 g; water, 1000 ml; pH 7.0±0.2, Difco Laboratories, Detroit, Mich.) at 37° C. were seeded on Mueller-Hinton plates by swab. Antibiotic-impregnated discs (6 mm diameter, BBL Sensi-Disc Susceptibility Test Discs; BD BBL Sensi-Disc Antimicrobial Discs) were placed on seeded plates and the zone of growth inhibition was measured following 18 hours of incubation at 37° C.

As shown in Table 4 below, the strains tested were found to be sensitive to most of the antibiotics currently used, such as ticarcillin, carbenicillin, imipenem, aminoglycosides etc. Interestingly, the Biosporin-derived strains (i.e., Bacillus subtilis HE and Bacillus licheniformis PA) differed in their sensitivity to a number of antibiotics. Thus, for example, Bacillus licheniformis PA was resistant to meticillin and oxacillin, whereas B. subtilis HE was sensitive to these antibiotics. The same differences were evident for ampicillin, benzylpenicillin, ceftazidim, clindamycin, and polymyxin E. Both strains were resistant to Aztreonam and Cefuroxim.

TABLE 4

Zone of inhibition (mm)

| Antibiotic | B. subtilis HE | B. licheniformis PA |
|---|---|---|
| Azlocillin | 18 ± 0.2 | 16 ± 0.1 |
| Amoxicillin | 18 ± 0.1 | 16 ± 0.1 |
| Ampicillin | 10 ± 0.3 | 0 |
| Carbenicillin | 24 ± 0.4 | 18 ± 0.3 |
| Mezlocillin | 20 ± 0.3 | 17 ± 0.2 |
| Methicillin | 18 ± 0.1 | 0 |
| Oxacillin | 14 ± 0.3 | 0 |
| Benzylpenicillin | 8 ± 0.1 | 0 |
| Piperacillin | 18 ± 0.3 | 12 ± 0.1 |
| Ticarcillin | 24 ± 0.4 | 20 ± 0.1 |
| Aztreonam | 0 | 0 |
| Imipenem | 36 ± 0.4 | 32 ± 0.3 |
| Moxalactam | 12 ± 0.3 | 12 ± 0.3 |
| Cephalothin | 32 ± 0.5 | 20 ± 0.4 |
| Cefazolin | 24 ± 0.2 | 20 ± 0.1 |
| Cefamandol | 37 ± 0.1 | 16 ± 0.1 |
| Cefoxitin | 16 ± 0.3 | 12 ± 0.3 |
| Cefoperazon | 16 ± 0.1 | 12 ± 0.2 |
| Cefotaxim | 14 ± 0.2 | 10 ± 0.2 |
| Ceftazidim | 8 ± 0.2 | 0 |
| Ceftizoxim | 0 | 0 |
| Ceftriaxon | 18 ± 0.3 | 12 ± 0.2 |
| Cefuroxim | 0 | 0 |
| Amikacin | 20 ± 0.2 | 18 ± 0.1 |
| Gentamycin | 24 ± 0.2 | 20 ± 0.1 |
| Kanamycin | 23 ± 0.1 | 20 ± 0.1 |
| Tobramycin | 24 ± 0.3 | 20 ± 0.2 |
| Vancomycin | 12 ± 0.1 | 12 ± 0.1 |
| Clindamycin | 10 ± 0.3 | 0 |
| Tetracycline | 24 ± 0.3 | 24 ± 0.2 |
| Chloramphenicol | 16 ± 0.2 | 10 ± 0.1 |
| Polymyxin E | 10 ± 0.1 | 0 |
| Nitrofurantoin | 16 ± 0.2 | 18 ± 0.1 |
| Trimethoprim | 24 ± 0.1 | 24 ± 0.1 |
| Bactrim | 30 ± 0.2 | 30 ± 0.1 |
| Norfloxacin | 24 ± 0.2 | 24 ± 0.2 |

### Example 5

### Antagonistic Activity of Probiotic Strains Bacillus subtilis HE and Bacillus licheniformis PA

Materials and Experimental Procedures

Bacterial preparation—Lot I—Probiotic strains B. subtilis HE and B. licheniformis PA were grown separately on Nutrient Agar for 24-48 hours at 37° C. All cultures were harvested in saline and diluted to a density of 10⁹ CFU/ml. B. subtilis HE and B. licheniformis PA were mixed in 3:1 ratio, respectively. A stabilizer including 1% gelatin and 4% saccharose was added and the mixture was poured into the ampoules and dried lyophilically.

Lot II—Probiotic strains B. subtilis HE and B. licheniformis PA were grown and harvested as described hereinabove but diluted to a density of 10¹⁰ CFU/ml.

Lot III—Probiotic strains B. subtilis HE and B. licheniformis PA were grown and harvested as described hereinabove but diluted to a density of 10¹¹ CFU/ml.

Antimicrobial activity assay—Antimicrobial activity of the bacterial strains of the present invention was assayed as described in Sorokulova et al., (1997) J. Travel Med. 4, 167-170 and Pinchuk (2001) Antimicrob Agents Chemother; 45(11):3156-61. In brief, each probiotic strain was inoculated as a spot (approximately 5-10 mm) on the surface of Mueller Hinton agar plates (Difco Laboratories, Detroit, Mich.). Following 72 h at 30° C., the bacteria were killed by exposure to chloroform vapor as described hereinabove. The inoculum of the test-cultures were prepared in order to contain approximately 10⁷ CFU/ml. These suspensions were streak plated from the border of the Bacillus spot to the edge of the plate. Plates were incubated for 24 h at 37° C. aerobically. An antagonistic activity of probiotic was indicated by the presence of inhibition zones of test-cultures.

Results

Table 5 below, lists the antipathogenic activity of the probiotic strains of the present invention in comparison to Biosporin. Evidently, the probiotic strains of the present invention mediated higher antagonistic activity against pathogenic and potentially pathogenic microorganisms in comparison with Biosporin (10⁹ CFU/ml).

TABLE 5

Antagonistic activity against
test-cultures (mm)

| | B. subtilis HE + | | | |
|---|---|---|---|---|
| | B. licheniformis PA | | | |
| Test-cultures | I | II | III | Biosporin |
| Salmonella typhimurium | 15 | 18 | 20 | 11 |
| S. typhi | 17 | 20 | 21 | 8 |
| Shigella sonnei | 18 | 17 | 19 | 16 |
| S. flexneri | 25 | 27 | 26 | 24 |
| Staphylococcus aureus | 30 | 33 | 31 | 25 |
| Klebsiella pneumoniae | 13 | 15 | 14 | 10 |
| E. coli 0157:H7 | 20 | 19 | 22 | 15 |
| Proteus vulgaris | 22 | 25 | 23 | 19 |
| Candida albicans | 34 | 35 | 32 | 30 |

### Example 6

### Antagonistic Activity of Probiotic Strains Bacillus subtilis HE and Bacillus licheniformis PA Against Enteropathogenic Strains of E. coli O157.H7

Experimental Procedures

As described in Example 5 above.

Results

As shown in Table 6, below, a higher antipathogenic activity of the probiotic strains of the present invention against enteropathogenic strains of E. coli O157:H7 was detected as compared to Biosporin. Note, a 1.6 fold higher activity of the bacterial strains of the present invention against strain 10* of E. coli O157:H7 as compared to Biosporin.

TABLE 6

| Strains of E. coli | Growth inhibition zones of test-cultures (mm) | | | |
|---|---|---|---|---|
| 0157:H7 | Lot I | Lot II | Lot III | Biosporin |
| 10-III | 16.1 | 15.9 | 16.0 | 13.7 |
| 10* | 11.3 | 11.5 | 11.4 | 7.0 |
| 23-III | 15.8 | 15.9 | 15.8 | 14.7 |
| 120-III | 12.0 | 12.2 | 11.9 | 10.5 |
| 4 | 7.5 | 6.9 | 7.3 | 5.4 |
| 1282 | 16.0 | 16.1 | 15.7 | 14.3 |
| 904 | 17.8 | 17.5 | 18.0 | 16.0 |
| 1330 | 16.6 | 17.0 | 17.4 | 15.0 |
| 9-III | 14.9 | 15.1 | 14.8 | 12.5 |
| 12 | 14.5 | 14.5 | 13.9 | 12.0 |
| 20 | 15.7 | 15.8 | 15.6 | 14.2 |
| 122 | 16.0 | 16.2 | 15.9 | 14.4 |
| 61-III | 17.4 | 16.9 | 17.4 | 15.8 |
| 13 | 15.2 | 15.6 | 15.3 | 13.0 |
| 18-III | 10.6 | 10.7 | 10.9 | 9.5 |
| III | 15.3 | 15.5 | 15.2 | 13.8 |

### Example 7

### In-Vivo Antagonistic Activity of Probiotic Strains Bacillus subtilis HE and Bacillus licheniformis PA Against Enteropathogenic Strains of E. coli O157:H7

Experimental Procedures

Antagonistic activity of lots I-III was studied on the mice with experimental E. coli O157:H7 infection. The mice were treated intraperitoneally with E. coli O157:H7 (strain 212)—1×10⁹ CFU per mouse. One day after injection of E. coli O157:H7 mice were orally treated with probiotics during four days (once a day). Control mice Were administered with saline.

Results

TABLE 7

| Days after | Defense of mice treated with probiotics, % | | | | |
|---|---|---|---|---|---|
| treatment with | | | | | Control |
| E. coli | Lot I | Lot II | Lot III | Biosporin | (saline) |
| 1 | | | | | 80 |
| 2 | 80 | 80 | 80 | 60 | 20 |
| 3 | 70 | 70 | 70 | 50 | 20 |
| 4 | 60 | 60 | 60 | 50 | 20 |
| 5 | 60 | 60 | 60 | 50 | 20 |

### Example 8

### Antagonistic Activity of Probiotic Strains Bacillus subtilis HE and Bacillus licheniformis PA Against Vaccine Strain of Bacillus anthracis

Background and Results

Anthrax is a naturally occurring disease among animals that ingest the bacterium Bacillus anthracis. The disease is quite common in agricultural regions where it occurs in animals. These include South and Central America, Southern and Eastern Europe, Asia, Africa, the Caribbean, and the Middle East. When anthrax affects humans, it is usually due to an occupational exposure to infected animals or their products. Workers who are exposed to dead animals and animal products from other countries where anthrax is more common may become infected with B. anthracis (industrial anthrax). Anthrax in wild livestock has occurred in the United States.

Anthrax infection can occur in three forms: cutaneous (skin), inhalation, and gastrointestinal. B. anthracis spores can live in the soil for many years, and humans can become infected with anthrax by handling products from infected animals or by inhaling anthrax spores from contaminated animal products. Anthrax can also be spread by eating undercooked meat from infected animals. It is rare to find infected animals in the United States.

Symptoms of disease vary depending on how the disease was contracted, but symptoms usually occur within 7 days.

Cutaneous—Most (about 95%) anthrax infections occur when the bacterium enters a cut or abrasion on the skin, such as when handling contaminated wool, hides, leather or hair products (especially goat hair) of infected animals. Skin infection begins as a raised itchy bump that resembles an insect bite but within 1-2 days develops into a vesicle and then a painless ulcer, usually 1-3 cm in diameter, with a characteristic black necrotic (dying) area in the center. Lymph glands in the adjacent area may swell. About 20% of untreated cases of cutaneous anthrax will result in death. Deaths are rare with appropriate antimicrobial therapy.

Inhalation—Initial symptoms may resemble a common cold. After several days, the symptoms may progress to severe breathing problems and shock. Inhalation anthrax is usually fatal.

Intestinal—The intestinal disease form of anthrax may follow the consumption of contaminated meat and is characterized by an acute inflammation of the intestinal tract. Initial signs of nausea, loss of appetite, vomiting, fever are followed by abdominal pain, vomiting of blood, and severe diarrhea. Intestinal anthrax results in death in 25% to 60% of cases.

The debate surrounding the anthrax vaccine is quite vast. Not only do its dangers outweigh the benefit but also the creator, BioPort of Lansing, Mich., has been halted by the FDA due to major manufacturing violations. The United States military recently stopped their vaccination efforts due to illness and deaths related to the inoculation. Thus, the vaccine is not a viable option for the general population any time soon.

Antagonistic activity of the probiotic culture of the present invention against the vaccine strain of Bacillus anthracis was tested as described in Example 4.

Results are summarized in Table 8, below.

TABLE 8

| | Zones of growth inhibition of test-cultures, mm | | | |
|---|---|---|---|---|
| Test-cultures | Lot I | Lot II | Lot III | Biosporin |
| Bacillus anthracis | 15.3 | 16.1 | 15.9 | 14.5 |

These results show for the first time that probiotics may be used for treating anthrax and as such may be a valuable replacement of the currently available vaccine.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A biologically pure culture of a bacterial strain having all the identifying characteristics of the Bacillus subtilis HE strain (ATCC Deposition No: PTA-5310).

2. A biologically pure culture of a bacterial strain having all the identifying characteristics of the Bacillus licheniformis PA strain (ATCC Deposition No: PTA-5311).

3. A bacterial co-culture comprising a first bacterial strain having all the identifying characteristics of Bacillus licheniformis PA (ATCC Deposition No: PTA-5311) and a second bacterial strain having all the identifying characteristics of Bacillus subtilis HE (ATCC Deposition No: PTA-5310).

4. A bacterial co-culture comprising at least two bacterial strains including a Bacillus licheniformis strain and a Bacillus subtilis strain, the bacterial co-culture exhibiting a higher anti-pathogenic activity than a Biosporin culture.

5. The bacterial co-culture of claim 4, wherein said Bacillus licheniformis strain is Bacillus licheniformis PA (ATCC Deposition No: PTA-5311) and said Bacillus subtilis strain is Bacillus subtilis HE (ATCC Deposition No: PTA-5310).

6. A composition comprising a therapeutically effective amount of a first bacterial strain having all the identifying characteristics of Bacillus licheniformis PA (ATCC Deposition No: PTA-5311) and/or a second bacterial strain having all the identifying characteristics of Bacillus subtilis HE (ATCC Deposition No: PTA-5310) and a pharmaceutically acceptable carrier.

7. The composition of claim 6 including at least 10³ viable bacteria cells per gram.

8. The composition of claim 6 including at least 10⁶ viable bacteria cells per gram.

9. The composition of claim 6 including at least 10¹⁰ viable bacteria cells per gram.

10. The composition of claim 6, wherein said first or said second bacterial strain is provided in a sporolated form.

11. The composition of claim 6, wherein said first or said second bacterial strain is provided in a lyophilized form.

12. The composition of claim 6, further comprising a probiotic microorganism selected from the group consisting of a yeast cell, a mold and a bacterial cell.

13. The composition of claim 6 further comprising an antibiotic.

14. The composition of claim 6 further comprising an antifungal agent.

15. A food additive or supplement comprising an effective amount of a first bacterial strain having all the identifying characteristics of Bacillus licheniformis PA (ATCC Deposition No: PTA-5311) and/or a second bacterial strain having all the identifying characteristics Bacillus subtilis HE (ATCC Deposition No: PTA-5310) and a carrier suitable for human consumption.

16. The food additive of claim 15, wherein said carrier is a colonization carrier.

17. The food additive of claim 16, wherein said colonization carrier is selected from the group consisting of a saccharide, a modified saccharide and a combination thereof.

18. The food additive of claim 15, wherein said first or said second bacterial strain is provided in a sporolated form.

19. The food additive of claim 15, wherein said first or said second bacterial strain is provided in a lyophilized form.

20. A feed additive or supplement comprising an effective amount of a first bacterial strain having all the identifying characteristics of Bacillus licheniformis PA (ATCC Deposition No: PTA-5311) and/or a second bacterial strain having all the identifying characteristics of Bacillus subtilis HE (ATCC Deposition No: PTA-5310) and a carrier suitable for animal consumption.

21. The feed additive of claim 20, wherein said carrier is selected from the group consisting of limestone, saccharides and wheat midds.

22. The feed additive of claim 20, wherein said first or said second bacterial strain is provided in a sporolated form.

23. The feed additive of claim 20, wherein said first or said second bacterial strain is provided in a lyophilized form.

24. A foodstuff comprising an effective amount of a first bacterial strain having all the identifying characteristics of Bacillus licheniformis PA (ATCC Deposition No: PTA-5311) and/or a second bacterial strain having all the identifying characteristics of Bacillus subtilis HE (ATCC Deposition No: PTA-5310).

25. The foodstuff of claim 24, being a fermented milk product.

26. The foodstuff of claim 24, wherein said first or said second bacterial strain is provided in a sporolated form.

27. The foodstuff of claim 24, wherein said first or said second bacterial strain is provided in a lyophilized form.

28. A method of treating or preventing a gastrointestinal disorder, the method comprising administering to a subject in need thereof a therapeutically effective amount of a first bacterial strain having all the identifying characteristics of Bacillus licheniformis PA. (ATCC Deposition No: PTA-5311) and/or a second bacterial strain having all the identifying characteristics of Bacillus subtilis HE (ATCC Deposition No: PTA-5310).

29. The method of claim 28, wherein said first or said second bacterial strain is provided in a sporolated form.

30. The method of claim 28, wherein said first or said second bacterial strain is provided in a lyophilized form.

31. The method of claim 28, wherein said administering is effected at a concentration of said first bacterial strain and/or said second bacterial strain between 10⁸ and 10¹⁰ viable cells in one dose.

32. An article-of-manufacture comprising packaging material and a composition identified for treating or preventing a gastrointestinal disorder being contained within said packaging material, said composition including, as an active ingredient, a first bacterial strain having all the identifying characteristics of Bacillus licheniformis PA (ATCC Deposition No: PTA-5311) and/or a second bacterial strain having all the identifying characteristics of Bacillus subtilis HE (ATCC Deposition No: PTA-5310).

33. The article-of-manufacture of claim 32, wherein said first or said second bacterial strain is provided in a sporolated form.

34. The article-of-manufacture of claim 32, wherein said first or said second bacterial strain is provided in a lyophilized form.

35. A method of treating or preventing a disorder which may be treated or prevented by probiotics, the method comprising administering to a subject in need thereof a therapeutically effective amount of a first bacterial strain having all the identifying characteristics of Bacillus licheniformis PA (ATCC Deposition No: PTA-5311) and/or a second bacterial strain having all the identifying characteristics of Bacillus subtilis HE (ATCC Deposition No: PTA-5310).

36. The method of claim 35, wherein said first or said second bacterial strain is provided in a sporolated form.

37. The method of claim 35, wherein said first or said second bacterial strain is provided in a lyophilized form.

38. The method of claim 35, wherein said administering is effected at a concentration of said first bacterial strain and/or said second bacterial strain between 10⁸ and 10¹⁰ viable cells in one dose.

39. The method of claim 35, wherein the disorder is selected from the group consisting of appendicitis, autoimmune disorders, multiple sclerosis, Alzheimer's disease, rheumatoid arthritis, coeliac disease, diabetes mellitus, organ transplantation, periodontal disease, urogenital diseases, sexually transmitted disease, HIV infection, HIV replication, surgical associated trauma, surgical-induced metastatic disease, sepsis, weight loss, anorexia, fever control, cachexia, wound healing, ulcers, gut barrier function, allergy, asthma, respiratory disorders, rhinovirus-associated diseases, circulatory disorders, coronary heart disease, anaemia, disorders of the blood coagulation system, renal disease, disorders of the central nervous system, hepatic diseases, constipation, ischaemia, nutritional disorders, osteoporosis, endocrine disorders, epidermal disorders, psoriasis, anthrax and acne vulgaris.
